(19) United States
(12) Patent Application Publication
(10) Pub. No.: US 2007/0049973 A1
(43) Pub. Date: Mar. 1, 2007

(54) Method and device for treating adenomyosis and endometriosis

(75) Inventors: Fred H. Burbank, Laguna Niguel, CA (US); Michael L. Jones, San Clemente, CA (US); R. J. Serra, Irvine, CA (US); Greig E. Altieri, Laguna Beach, CA (US); Jill Uyeno, Mission Viejo, CA (US); Yu-Tung Wong, Cleveland Heights, OH (US); Randy Werneth, San Diego, CA (US)

Correspondence Address: Edward J. Lynch / Duane Morris LLP;One Market, 20th Floor, Spear Tower, San Francisco, CA, 94105, US

(73) Assignee: Vascular Control Systems, Inc.

(21) Appl. No.: 11/214,606

(22) Filed: Aug. 29, 2005

(51) Int. Cl. A61N 1/00

(52) U.S. Cl. 607/2

(57) ABSTRACT

The invention provides devices, systems and methods for reducing or abolishing blood flow by occluding uterine arteries for treating adenomyosis and endometriosis. A non-invasive uterine artery occlusion device embodying features of the invention includes a pair of pressure-applying members with opposed tissue-contacting surfaces, a supporting shaft configured to adjust the distance between tissue-contacting surfaces, and at least one sensor for locating a uterine artery disposed on at least one pressure-applying member. Uterine arteries are occluded by indirectly compressing the artery by compressing tissue near to an artery. One uterine artery may be occluded or both may be occluded simultaneously. A uterine artery may be accessed via a body cavity, such as a patient's vagina, and may be occluded by compressing a portion of the vaginal wall around a portion of a uterine artery.

### RELATED APPLICATIONS

This application relates to and claims priority of U.S. patent application Ser. No. 09/556,934 filed Apr. 21, 2000, now U.S. Pat. No. 6,550,482, U.S. patent application Ser. No. 10/300,115 filed Nov. 19, 2002, U.S. patent application Ser. No. 09/909,815 filed Jul. 20, 2001, U.S. patent application Ser. No. 10/113,096 filed Mar. 28, 2002, U.S. patent application Ser. No. 10/107,810 filed Mar. 28, 2002, U.S. patent application Ser. No. 11/151,808, filed Jun. 13, 2005 which is a continuation of application Ser. No. 10/107,810, and provisional application 60/279,477 filed Mar. 28, 2001. Each of the applications are incorporated by reference in their entirety.

### FIELD OF THE INVENTION

The invention relates generally to the field of treatment of diseases and conditions by the regulation of blood flow in blood vessels. In particular, the invention is directed to the treatment of uterine disorders by detecting and regulating blood flow thereto.

### BACKGROUND OF THE INVENTION

Hysterectomy (surgical removal of the uterus) is performed on approximately 600,000 women annually in the United States. For approximately 340,000 women, hysterectomy is probably the best current therapeutic choice for the treatment of their uterine diseases and disorders, such as cancer, endometriosis, adenomyosis, menorrhagia, and prolapse. For approximately 60,000 women with dysfunctional uterine bleeding (abnormal menstrual bleeding that has no discrete anatomic explanation such as a tumor or growth), newer endometrial ablation techniques may be an alternative to hysterectomy. For approximately 200,000 women with benign but symptomatic (excessive bleeding, pain, and “bulk” sensations) muscular tumors of the uterus, known as leiomyoma or fibroids, newer treatment methods have been developed which may spare these women a hysterectomy, as well.

However, hysterectomy is a drastic treatment, having many undesirable characteristics. Thus, any method which can approximate the therapeutic result of a hysterectomy without removing the uterus would be a significant improvement in this field. Newer treatment methods have been developed for some diseases which may spare these women a hysterectomy.

In 1995, it was demonstrated that uterine fibroids could be treated without hysterectomy using a non-surgical therapy, specifically comprising bilateral intraluminal occlusion of the uterine arteries (Ravina et al., “Arterial Embolization to Treat Uterine Myomata”, Lancet Sep. 9, 1995; Vol. 346; pp. 671-672, incorporated in its entirety herein). This technique is known as “uterine artery embolization”. In this technique, uterine arteries are accessed via a transvascular route from a common femoral artery into the left and right uterine arteries.

The uterus has a dual (or redundant) blood supply, the primary blood supply being from the bilateral uterine arteries, and the secondary blood supply from the bilateral ovarian arteries. Consequently, when both uterine arteries are occluded, i.e. bilateral uterine artery occlusion, the uterus and the fibroids contained within the uterus are both deprived of their blood supply. However, as demonstrated by Ravina et al., the effect on the fibroid is greater than the effect on the uterus. In most instances, the fibroid withers and ceases to cause clinical symptoms.

However, many physicians do not possess the skill or equipment necessary to perform catheter-based uterine artery embolization under radiologic direction. Accordingly, only thousands of uterine artery embolizations have been performed, worldwide, over the past three years, whereas hundreds of thousands of hysterectomies have been performed each year for uterine fibroids which are symptomatic.

What is needed, therefore, are better devices and methods to treat uterine disorders that can be used by physicians of ordinary skill in a simple medical setting or environment.

### SUMMARY OF THE INVENTION

The invention is directed to non-invasive devices, systems and methods for extravascularly detecting blood flow in a uterine artery, and for occluding a uterine artery effective to reduce or abolish blood flow in it to treat adenomyosis and endometriosis. The non-invasive devices, systems and methods embodying features of the invention are configured to be non-surgically applied externally of a uterine artery which they occlude, and are preferably applied at least in part extracorporeally. The occlusion is temporary, and may be partial or complete. One method of occluding a uterine artery comprises clamping the uterine artery effective to compress it so that blood flow through the artery is reduced, or is abolished. Such clamping of a uterine artery may be direct or may be indirect. Preferably, clamping of a uterine artery effective to compress it is accomplished by applying a non-invasive uterine artery occlusion device to tissue near to a uterine artery (e.g., onto tissue surrounding the artery). A uterine artery occlusion device may also be applied directly onto a uterine artery effective to compress the uterine artery.

In one embodiment of the invention, a non-invasive uterine artery occluding device (such as a clamp with a sensor) may be applied to a portion of a vaginal wall to detect and/or locate, and then to occlude the uterine arteries. A vaginal clamp embodying features of the invention is used to sense the location of a uterine artery adjacent a vaginal wall, and may be used to compress and occlude a uterine artery adjacent a vaginal wall. The vaginal wall may be distended by an occlusion device so as to more closely approach a uterine artery; such an approach may aided by applying pressure or force to the uterus (e.g., by pulling on the uterine cervix). A uterine cervix may be grasped or pulled by any suitable device or implement, including forceps, suction devices, and other instruments, such as a tenaculum.

A non-invasive uterine artery occluding device embodying features of the invention comprises a pair of pressure-applying members having opposed tissue-contacting surfaces on distal portions thereof; at least one supporting shaft extending from a proximal extremity of at least one of the pressure-applying members which is configured to adjust the distance between the opposed tissue-contacting surfaces of the pressure-applying members; and at least one blood flow sensing sensor on one of the opposed tissue-contacting surfaces. An embodiment of a non-invasive uterine artery occlusion device embodying features of the invention may have, for example, a handle, a clamping member configured to apply pressure or force to body tissue, and a sensor for locating a uterine artery.

A pressure-applying member, such as a clamping member, is, e.g., a jaw or jaws configured to engage a uterine artery or to engage tissue adjacent a uterine artery. A supporting shaft, such as a handle, is preferably configured for manipulating the jaw or jaws. In some embodiments of devices having features of the invention, a pressure-applying member is attached to a connecting portion that is configured so that a jaw may be placed within a vagina while a handle remains outside a patient's body and available for use by an operator.

A sensor for locating a uterine artery senses sound, pulsation, blood flow or other indicator related to a uterine artery. Thus, a sensor for locating a uterine artery may be a blood flow sensor, a sound sensor, a pressure sensor, a strain sensor, a stress sensor, a chemical sensor, an electromagnetic radiation sensor, or other sensor, and may be a combination of such sensors. A sound sensor may be an ultrasound sensor, including a Doppler ultrasound sensor. The sensor for locating a uterine artery, including a sensor for measuring blood flow, is preferably disposed in or on a pressure-applying member, and is preferably mounted to the face of a tissue-contacting surface, such as the face of a jaw of a clamp. A sensor is preferably oriented perpendicularly to the clamp face, although in embodiments of devices having features of the invention a sensor may assume other orientations.

A system embodying features of the invention includes a uterine artery occluding device having a pair of pressure-applying members configured to apply pressure or force to body tissue, at least one supporting shaft, a sensor for locating a uterine arterty, and a sensor controller which may include an energy source. A system may further include a device for grasping a portion of a patient's body, such as a device for grasping a uterine cervix.

A sensor controller is configured to aid in detecting a location of a uterine artery, by, e.g., providing a signal related to the output of a sensor that may be readily used by an operator. A sensor controller preferably includes an energy source configured to provide energy for operating a sensor for sensing a location of a uterine artery, such as ultrasound energy, electrical energy, or electromagnetic energy. The energy is directly provided by the energy source or is provided by the sensor with the aid of the energy source. Ultrasound energy useful for sensing a location of a uterine or of blood flow in a uterine artery may have a frequency of less than about 20 MegaHertz (MHz), such as between about 5 MHz and about 19 MHz, preferably between about 6 MHz and about 10 MHz, more preferably a frequency of about 8 MHz. Electromagnetic energy useful for sensing a location of a uterine artery or of blood flow in a uterine artery may have a wavelength of between about 500 nanometers (nm) and about 2000 nm, preferably between about 700 nm and about 1000 nm.

In one embodiment of the invention a non-invasive uterine artery occluding device embodying features of the invention includes a pair of pressure applying members having opposed tissue contacting surfaces on distal portions thereof and at least one blood flow sensing sensor on each of the opposed tissue contacting surfaces. Preferably the device is configured to be insertable through the vagina of a female patient, along a side of the exterior of the cervix, and to the vaginal wall at the vaginal fornix. The bilateral structures of the device permit both the left and right uterine arteries to be compressed at the same time upon upward pushing of the device, and using the body of the uterus as an anvil against which to compress the arteries.

A method for occluding a uterine artery includes locating a uterine artery with one or more sensors and compressing a portion of the uterine artery with a non-invasive uterine artery occluding device which includes the sensor. A method of occluding a uterine artery of a patient may include locating a uterine artery with a sensor and compressing a portion of the uterine artery with a non-invasive uterine artery occluding device which includes the sensor. Compressing a portion of a uterine artery may include applying pressure or force to a vaginal wall. In addition, methods for occluding a uterine artery include applying tension to a uterus and applying pressure or force to a vaginal wall, and include engaging a uterine cervix with a grasping implement (e.g., by pulling on the uterine cervix) while applying force or pressure to a vaginal wall to occlude a uterine artery.

Also methods for occluding uterine arteries include occluding both the left and right uterine arteries simultaneously by inserting the pressure applying members along the exterior of the cervix and to the vaginal wall at the vaginal fornix, then compressing both arteries against the uterus.

The non-invasive devices, systems and methods embodying features of the invention allow the non-surgical location and occlusion of uterine arteries, providing therapeutic temporary, partial or complete, reduction or abolition of blood flow in the located and occluded uterine arteries. Use of the devices, systems and methods of the present invention thus allow the occlusion of a uterine artery without the puncture of bodily tissue, and without the need for radiographic equipment or for skill in the use of radiographic techniques. The devices and methods are simpler and more readily used and removed than other methods and devices, and provide improved treatments for serious uterine disorders, including uterine fibroids, dysfunctional uterine bleeding (DUB), endometriosis, adenomyosis, post-partum hemorrhage, and other uterine disorders. The devices, systems and methods embodying features of the invention thus provide tools and methods for effective treatment of diseases and conditions that otherwise require invasive and irreversible treatments such as removal of a uterus.

### BRIEF DESCRIPTION OF THE DRAWINGS

FIG. 1 is a plan view of a system embodying features of the invention including a vaginal clamp embodying features of the invention disposed in an open configuration.

FIG. 2 is a fragmentary sectional view of a distal portion of a clamping device embodying features of the invention in a closed configuration.

FIG. 3 is a perspective view of a jaw portion of a vaginal clamp embodying features of the invention disposed in an open configuration.

FIG. 4 is a transverse cross-sectional view of a jaw portion of the clamping device of FIG. 3 taken at line 4-4.

FIG. 5 is schematic diagram of a reproductive system of a human female including major uterine arteries providing blood flow to the uterus.

FIG. 6 is a schematic diagram illustrating the use of a vaginal clamp embodying features of the invention in the occlusion a uterine artery of a female human patient.

FIG. 7A is is a plan view of a system embodying features of the invention including a vaginal clamp embodying features of the invention disposed in an open configuration.

FIG. 7B is a top plan view of a device embodying features of the invention.

FIG. 8A is a simplified schematic view of a uterus and a force vector.

FIG. 8B, 8C, and 8D schematically illustrate side elevational views of yet other embodiments in accordance with the present invention.

### DETAILED DESCRIPTION OF THE PREFERRED EMBODIMENTS

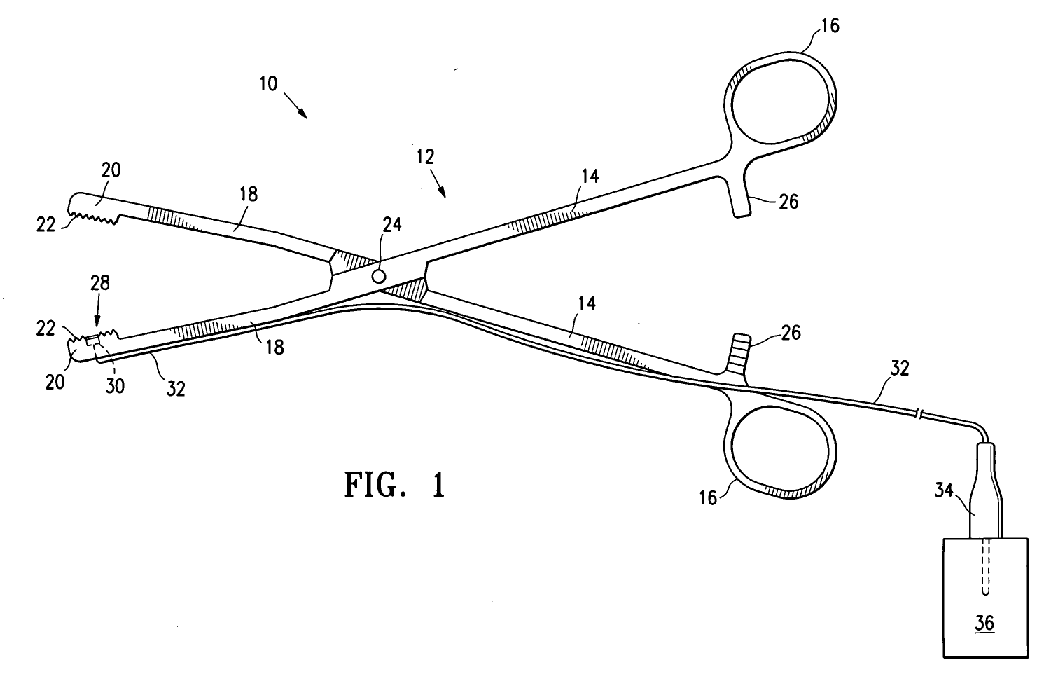

FIGS. 1-4 show a non-invasive uterine artery-occluding system 10 embodying features of the invention. The system 10 includes a clamping component 12, including handles 14, having finger holes 16, and pressure-applying members 18 with jaws 20 on their distal ends. Jaws 20 have serrated tissue-contacting surfaces 22 configured to engage and hold onto tissue when jaws 20 are pressed into a patient's body tissue. Pressure-applying members 18 are pivotally connected with each other at pivot point 24; handles 14 (which act as supporting shafts for device 12) are also integral with the pressure-applying members 18 and are pivotally connected with each other at pivot point 24. Squeezing handles 14 together, preferably by fingers of an operator's hand engaged with finger holes 16, is effective to cause tissue-contacting surfaces 22 to approach one another as pressure-applying members 18. Such motion provides mechanical advantage where the lengths of pressure-applying members 18 are not equal to the lengths of handles 14, allowing for greater or lesser force or pressure at tissue-contacting surfaces 22 than is applied at finger holes 16. For example, where the lengths of pressure-applying members 18 are less than the lengths of handles 14, greater force may be applied at tissue-contacting surfaces 22 than is applied at finger holes 16. Releasable ratcheting mechanism 26 includes two complementary portions configured to engage with each other and to lock handles 14 in a closed position, maintaining pressure or force between tissue-contacting surfaces 22 while the locking mechanism 26 is engaged.

A non-invasive uterine artery-occluding system 10 also includes a sensor component 28, such as a blood flow detection system, which includes a sensor 30 and a cable 32 having a proximal connector 34 configured to operably engage with a sensor control device 36. A connector 34 is preferably a releasable connector configured to readily engage and disengage with a sensor control device 36. Alternatively, a cable 32 directly and permanently engage a sensor control device 36 without having a connector 34. A sensor control device 36 may be configured to supply power that may be required by a sensor 30, to receive signals from a sensor 30, and to carry sensor signal outputs to a sensor controller for interpretation by an operator. A sensor 30 is a passive sensor (e.g., configured to detect intrinsic signals indicating the presence of a uterine artery) or active (e.g., configured to emit a signal, and to detect a signal in response to, or derived from, the emitted signal). An emitted signal is pulsed or continuous. A sensor controller 36 preferably produces and provides signals or signal energy used for sensing (e.g., ultrasound or infra-red signals or energy) or provides energy to a sensor 30 to aid the sensor 30 to produce or provide signals or signal energy. Cable 32 preferably includes an electrical cable, an optical fiber, a waveguide, other conduit for carrying energy or signals, or a combination of these.

A sensor 30 preferably is a blood flow sensor configured to identify and locate a uterine artery and for determining the degree of occlusion of the uterine artery. In particular, a sensor 30 preferably is configured to indicate the location of a uterine artery with respect to a jaw 20 of a device embodying features of the invention. A sensor 30 may thus be a blood flow sensor, but may also be a microphone (e.g., to sense heart sounds or other sounds not directly “blood flow” sounds, although turbulence due to flow may also produce detectable sounds), a pressure transducer or stress or strain gauge to detect pulsations in an artery due to heart action, a pH sensor, an electromagnetic radiation sensor, such as an infrared sensor, to detect a uterine artery (e.g., to detect hemoglobin), or other sensor. Preferably, sensor 30 is a Doppler ultrasound sensor, configured to emit and to detect ultrasound effective to detect blood flow and to locate a uterine artery.

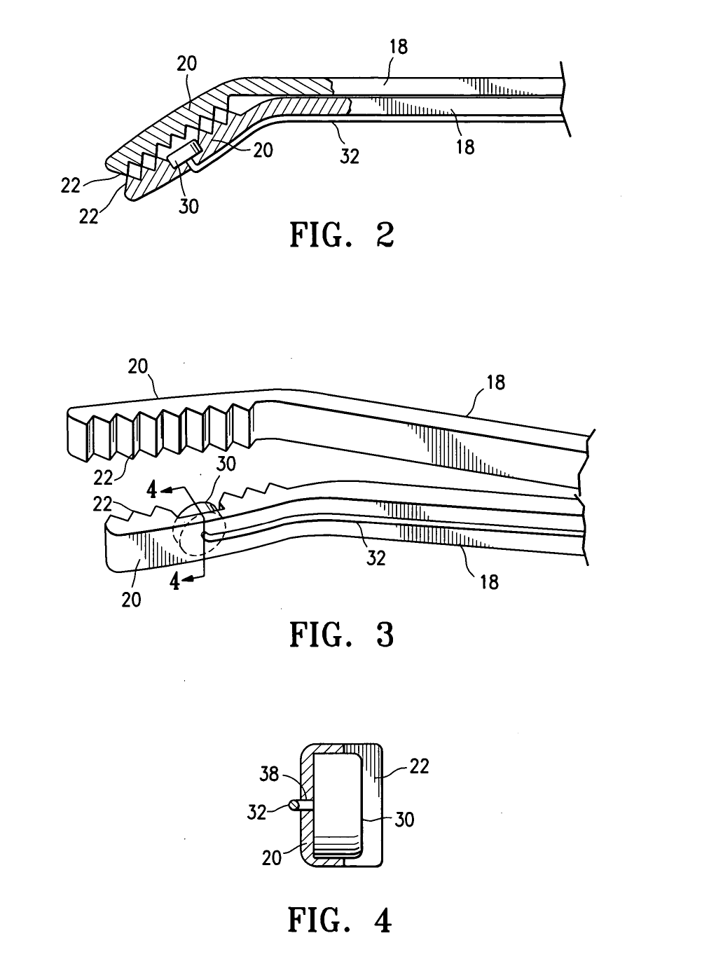

FIG. 2 illustrates a distal part of a system 10 embodying features of the invention, showing portions of pressure-applying members 18, and jaws 20 having tissue-contacting surfaces 22. In the embodiment shown in this figure, jaws 20 meet pressure-applying members 18 at an angle, unlike the embodiment shown in FIG. 1 where jaws 20 meet pressure-applying members 18 to form approximately straight angles. It will be understood that jaws 20 may be disposed at any suitable angle with respect to pressure-applying members 18. A sensor 30 on one jaw 20 is also shown, with a portion of cable 32 shown disposed along a portion of a pressure-applying member 18. Tissue-contacting surfaces 22 are shown in FIG. 2 disposed in close apposition to one another. Tissue-contacting surfaces 22 are placed in contact with tissue, including a portion of a uterine artery, disposed between jaws 20. Partial or complete closure of jaws 20 causes tissue-contacting surfaces 22 to apply pressure or force to the tissue effective to compress a uterine artery or the tissue around a uterine artery; the application of pressure or force is effective to compress the tissue and to occlude the uterine artery, reducing or abolishing blood flow through at least a portion of the uterine artery.

A sensor 30 is effective to detect the location of a uterine artery and to detect blood flow in a uterine artery. Such detection is used to direct a system 10 so as to ensure that body tissue including a portion of a uterine artery to be occluded is between jaws 20 of the clamping component 12. In preferred methods of use, the uterine artery and surrounding tissue is disposed between jaws 20 and pressure or force is applied to the tissue by tissue-contacting surfaces 22, applying pressure to the tissue, effective to compress a portion of a uterine artery and to at least partially occlude the uterine artery. Such compression and resulting occlusion of a uterine artery is effective to reduce or abolish blood flow in the artery. Sensor 30, disposed on jaws 20, may be effective to sense the reduction or abolition of blood flow in a compressed uterine artery.

FIG. 3 illustrates in greater detail the distal portion of a system 10 embodying features of the invention, showing a distal portion of a clamping component 12 having pressure-applying members 18 with jaws 20 having tissue-contacting surfaces 22. A sensor 30 is shown disposed on a jaw 20 on the tissue-contacting surface 22, with a distal portion of a cable 32 disposed opposite the tissue-contacting surface 22.

FIG. 4 is a cross-sectional view of a jaw 20, taken through a sensor 30 along line 4-4 of FIG. 3. The sensor 30 is connected with cable 32 by connection 38, which preferably is a wire, plurality of wires, optical fiber, waveguide, or other connection effective to carry signals and/or energy or power between a sensor 30, a cable 32, and a sensor controller 36. Preferably, connection 38 is a continuation of at least a portion of cable 32.

In one embodiment a sensor 30 is a blood flow sensor for locating a uterine artery, and is a passive sensor, configured to detect intrinsic signals indicating the presence of a uterine artery (i.e., a sound sensor, a motion sensor, a pH sensor, or other sensor configured to detect a physical, chemical, electrical, or physiological indication of the location of a uterine artery). In other embodiments, a blood flow sensor for locating a uterine artery is an active sensor, configured to emit energy or a signal, and configured to detect signals in response to, or derived from, the emitted energy or signal indicating the presence of a uterine artery (i.e., a source of ultrasound having an ultrasound sensor configured to detect ultrasound reflections from a uterine artery, a source of infrared radiation configured to detect reflections from a uterine artery, or other source of energy and a sensor configured to detect a response indicating the location of a uterine artery). The operation of a sensor may be aided by an energy source (which may be provided by a sensor controller 36), which may directly provide the energy detected by the sensor, or which may aid the sensor to provide the energy to be sensed. For example, an energy source may provide electrical energy which aids an ultrasound sensor to produce and to detect ultrasound energy (as, e.g., in the MedaSonics® CardioBeat® Blood Flow Doppler with Integrated Speaker (Cooper Surgical, Inc., Trumbull Conn. 06611)). Other commercially available Doppler ultrasound sensors suitable for use in the present invention include the Koven model ES 100X MiniDop VRP-8 probe (St. Louis, Mo.) and the DWL/Neuro Scan Medical Systems' Multi-Dop B+ system (Sterling, Va.).

Non-invasive uterine artery occluding devices embodying features of the invention include clamping devices having a pressure-applying member configured to apply pressure or force to a uterine artery and a blood flow sensor. A pressure-applying member preferably has a distal portion configured to engage tissue. Non-invasive uterine artery occluding devices embodying features of the invention preferably has two, or more, pressure-applying members. Two pressure-applying members preferably are disposed opposite each other and configured to move and/or to apply pressure or force towards each other, such as to close together, effective to engage tissue and to clamp a uterine artery between them. Alternatively, a pressure-applying member may have two portions disposed in apposition to one another, effective to clamp tissue between the portions.

Partial or total closure of a uterine artery, is effected by pressure applied through a body wall, such as the vaginal mucosa. Sufficient pressure or force applied to tissue is effective to apply pressure to that tissue and to underlying tissues and so to compress and to at least partially occlude a uterine artery. An amount of pressure applied through a body wall to effect closure of a uterine artery may be between about 15 pounds per square inch (psi) and about 125 psi, and preferably is between about 30 psi and about 60 psi. For example, where the pressure-applying surface has a surface area of about 0.16 square inches (e.g., a surface with dimensions of about 0.2 inches by about 0.8 inches), the amount of force applied by a non-invasive artery occluding device embodying features of the invention is preferably between about 3 pounds and about 20 pounds, and more preferably between about 6 pounds and about 9 pounds.

A sensor for detecting or locating a uterine artery may be any sensor configured to detect a uterine artery in place within body tissue. Such a sensor may detect sound, such as heart sounds, or other sounds intrinsically associated with uterine arteries. Alternatively, a sensor for locating an artery may produce or be associated with artificially created light or sound, such as ultrasound, and detect reflections or other signals derived from the artificially-produced light or sound. In preferred embodiments, a sensor is a blood flow sensor. A blood flow sensor, such as a Doppler blood flow sensor, may be disposed perpendicular to the tissue-contacting surface 22 of a jaw 20, effective that only arteries facing a jaw 20, or within the jaws 20, are detected by the blood flow sensor.

A sensor may detect a uterine artery, or blood flow, or signals related to the location of a uterine artery or of blood flow, in a particular direction. For example, a sensor disposed on a tissue-contacting surface of a pressure-applying member, such as a jaw of a clamp, may detect signals from a direction perpendicular to the surface of the jaw, and so be effective to locate uterine arteries or detect blood flow opposite the jaw. Such an orientation is effective to insure that a uterine artery to be occluded is positioned opposite a jaw, and between a pair of jaws, and so is properly placed for occlusion. A sensor may also be configured to detect signals from directions parallel to a tissue-contacting surface, or at some other angle with respect to a tissue-contacting surface; such configurations are useful, for example, for directing the movement of a non-invasive artery occluding device towards a uterine artery.

A blood flow sensor preferably includes Doppler ultrasound sensor. A blood flow sensor may be disposed on a clamping member, preferably on a distal portion configured to engage tissue, more preferably near the middle of the distal portion. A blood flow sensor disposed on a pressure-applying member preferably is configured to detect blood flow in a uterine artery near to the pressure-applying member, and may be configured to detect blood flow in a uterine artery clamped by a pressure-applying member or between pressure-applying members. Non-invasive uterine artery occluding devices embodying features of the invention may include more than one blood flow sensor. Preferred blood flow sensor include Doppler ultrasound blood flow sensors and near infrared blood flow sensors.

In one embodiment a non-invasive uterine artery occluding device embodying features of the invention is configured to lock into a clamping position. Such a locked configuration is temporary and releasable, or is permanent. Non-invasive uterine artery occluding devices embodying features of the invention preferably have a locking mechanism, such as a ratchet, configured to hold at least one pressure-applying member in a pressure-applying position. Such locking mechanisms preferably include a release mechanism effective to allow the cessation of pressure or force application when desired. Thus, a non-invasive uterine artery occlusion device embodying features of the invention preferably is configured to release a locking mechanism effective to relieve the occlusion of a uterine artery by ending the application of pressure or force that had been previously applied to occlude a uterine artery.

The apparatus and systems of the present invention are configured for use within a body cavity and for use adjacent a patient's skin or other body surface, but are non-invasive and configured for external use. Clamping devices may be of any suitable size, which is determined in part by the location and dimension of the artery to be occluded. The handle, jaws, and if present, connecting portion, are configured to allow access to tissue adjacent a uterine artery such as a uterine artery and to provide a clamping pressure or force to the tissue sufficient to occlude the uterine artery to reduce or abolish blood flow in it.

For accessing and occluding a uterine artery, the dimensions of a vagina help to determine suitable sizes for clamping devices and clamp applicators embodying features of the invention so that at least a portion of a vaginal clamp is configured to fit within a vagina, and can readily reach the vaginal fornix when operated from outside of a patient's body. For example, a clamping device may be between about 0.5 inch and about 16 inches in length, preferably between about 1 inch and about 12 inches in length.

Apparatus and systems configured for detecting and occluding blood flow embodying features of the invention are configured to invaginate vaginal mucosa when disposed within a vagina near to a uterine artery. Such apparatus and systems are configured to invaginate vaginal mucosa without puncturing a vaginal wall; that is, Without passing through the vaginal mucosa. A sensor may be configured, for example, to detect blood flow in an artery such as a uterine artery without puncturing a patient's skin or mucosal surface. A jaw or jaws of a device and of a system embodying features of the invention are configured to compress tissue adjacent an artery such as a uterine artery without puncturing a patient's skin or mucosal surface. Thus, a vaginal clamp embodying features of the invention is effective to detect the location of an artery such as a uterine artery and to occlude it.

A vaginal clamp embodying features of the invention has a jaw or jaws configured to engage a uterine artery or to engage tissue adjacent a uterine artery, and has an ultrasound sensor, such as a Doppler ultrasound sensor, mounted in a jaw. A Doppler ultrasound sensor operating at ultrasound frequencies less than about 20 MHz, such as between about 5 MHz and about 19 MHz, preferably between about 6 MHz and about 10 MHz, more preferably at about 8 Hz, is suitable for detecting blood flow in an artery with apparatus embodying features of the invention. A sensor is preferably mounted to the face of the clamp jaw and oriented perpendicularly to the jaw face. For example, a blood flow sensor may be mounted between about 0.1″ and about 1″ from the distal tip of a clamp jaw, and is preferably mounted about 0.2 to about 0.6″, more preferably about 0.4″ from the distal tip of a clamp jaw. A clamp jaw may be configured to tightly engage tissue, i.e., may have a surface that is serrated, scored, roughened, coated with a rough material including sandpaper, or otherwise configured to grip tissue. For example, a clamp jaw may be serrated in order to obtain sufficient grip force to remain in position over a uterine artery when clamped onto vaginal mucosa. A non-invasive artery occluding device may have more than two jaws. Multiple jaws are preferably disposed approximately symmetrically about a central axis, and configured so that all jaws approach a central position when closed, so that, for example, three jaws may be oriented approximately 120° from each other and disposed to close to a central point effective to capture tissue between them.

The dimensions of a vaginal clamp embodying features of the invention are chosen to facilitate use within a vagina, and so that the clamp can readily reach the vaginal fornix when operated from outside of a patient's body.

A jaw or jaws may be configured to join with the connecting portion on a line substantially parallel to a line along the connecting portion, or may join at an angle to such a line. An angle between a jaw or jaws and a connecting portion may be acute or may be obtuse. In preferred embodiments, the connection between a jaw or jaws and a connecting portion or portions is a rigid connection; in some embodiments, a jaw may be an extension of a connecting portion, and both may be formed of a single piece of material.

In one embodiment the pressure applying members of the non-invasive uterine artery occluding device in accordance with the present invention are sized to be insertable through the vagina of a female human patient, along a side of the exterior of the cervix, and to the vaginal wall at the vaginal fornix. The bilateral structures of the non-invasive uterine artery occluding system permit both the left and the right uterine arteries 48 and 50 to be compressed at the same time upon upward pushing of the device 10, and using the body of the uterus as an anvil against which to compress the arteries.

The spacing between the portions of the jaws 20 which bare on the vaginal wall can be dimensioned to accommodate the urethra and bladder neck on the anterior side of the cervix, and the rectum on the posterior side of the cervix. That is, the distalmost compressing ends of the device 10 can optionally be sized, both in their circumferential length and their longitudinal depth, so that when the device 10 is used to compress the left and right uterine arteries of a female human patient, the urethra, bladder neck, and rectum are not compressed as much, or are not compressed at all, which can limit or eliminate complications with these structures.

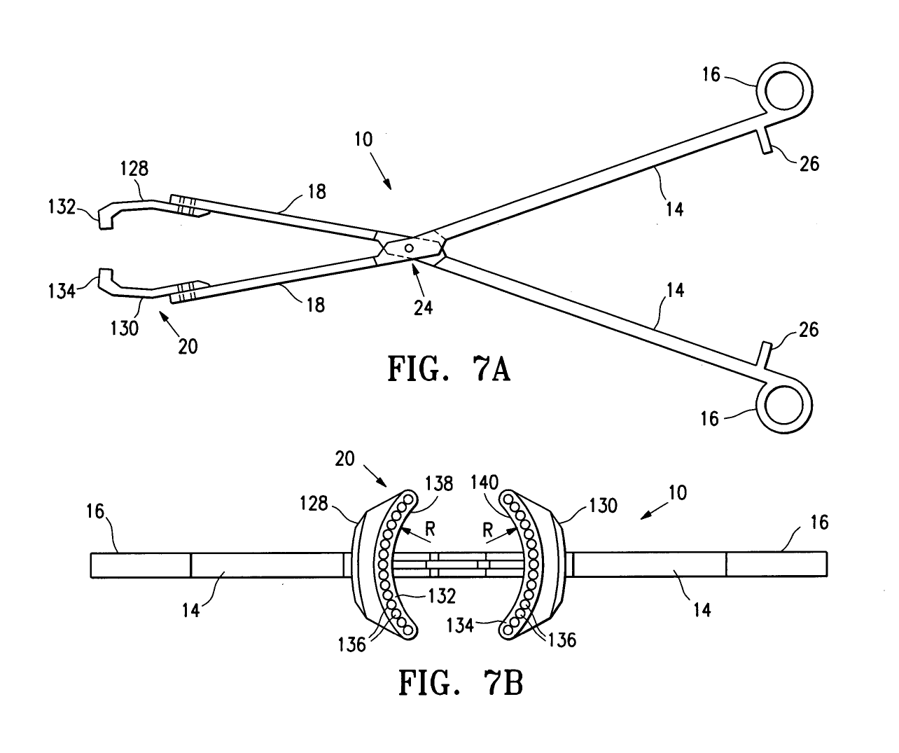

In the embodiment shown in FIGS. 7A and 7B the jaws 20 preferably includes at least one, and preferably two curved lateral interior surfaces 138, 140, one formed in each of the first and second parts 128, 130 of the jaws 20. The surfaces 138, 130 are preferably formed at a radius R. As suggested by the radius R, the curve of the jaws 20 can be semi-circular, but in general the curve is selected so that it approximates the shape of the exterior surface of the cervix at least where the cervix meets the vaginal fornix. By forming at least, a portion of jaws 20 with a concave inner surface 138, 140 which is similar in its curvature to the shape of the exterior surface of the cervix, the cervix itself can be used as a guide toward the uterine artery or arteries. That is, the jaws 20 can be pushed along the exterior of the cervix toward the uterine artery with the interior surface 138, 140 riding along the exterior of the cervix. In this manner, the orientation of the device 10 relative to the cervix and the uterine artery can be correctly maintained because the cervix acts as a rail on which the device 10 rides toward the uterine artery.

One or both of the first and second parts 128, 130 include at least one, and preferably a plurality or array of holes, bores, or channels 136 which are sized and configured to receive Doppler chips. Thus, when the device 10 includes the holes 136 and Doppler chips positioned therein, the device 10 can further be used to identify the location of a uterine artery of interest based upon its blood flow characteristics and monitor the blood flow through the uterine artery during the course of a procedure.

As the system is advanced upwardly along the cervix towards the uterus, as with prior embodiments described above, the uterine artery or arteries are entrapped between the uterine body and the device 10, and are compressed between the body of the uterus and the vaginal wall at the vaginal fornix; in turn, the vaginal wall is pushed by the distal end faces 132, 134, of the device 10.

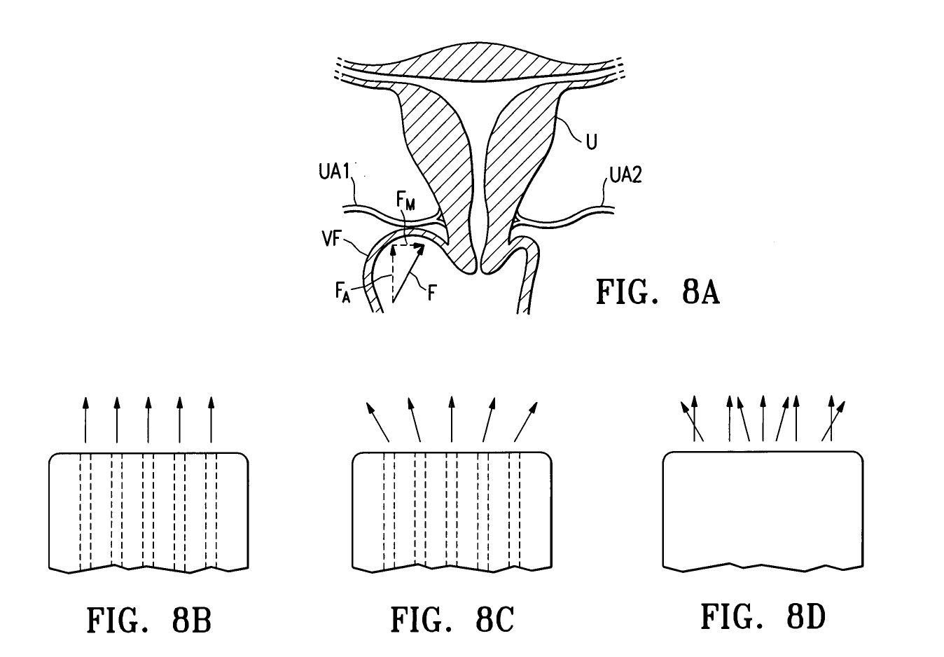

Further optionally, the system, and in particular, the finger rings 16, can be manipulated to move the first and second parts 128, 130 of the jaws 20 toward one another, thereby moving the entrapped uterine arteries toward the body of the uterus and additionally compressing the uterine arteries. At this point, one or more of the distal end faces 132, 134 and the lateral surfaces 138, 140 include the surfaces which transmit force from the device 10 to the patient's tissues. As will be readily appreciated by those of skill in the art, and schematically illustrated in FIG. 8A, the direction of the force applied against the uterine artery or arteries can include axial (parallel to the orientation of the cervix) and/or lateral (perpendicular to the orientation of the cervix) components.

In the embodiment shown in FIG. 8A the direction in which the compression force is applied against the vaginal fornix VF, and therefore against the uterine artery (UA1, UA2), includes at least an axial component F_{A}. According to other aspects of the present invention, the force vector of the force which generates the compression of the uterine artery can include a medial component F_{M}, i.e., the compression force vector F is also directed inward toward the centerline of the uterus U. According to yet further aspects of the present invention, the force vector F can be built by serially applying: an axial force F_{A}, and then a medial force F_{M}; a medial force F_{M}, and then an axial force :F_{A} or simultaneous combinations of axial F_{A} and medial F_{M} forces of various magnitudes. The addition of the medial force F_{M} component of the force vector F can assist in trapping or pinning the uterine artery against the uterus U when the uterus is used as an ‘anvil’ against which the uterine artery is compressed. According to the aspect of the invention in which the medial force F_{M} component of the force vector F is used, at least in part to compress a uterine artery, the distal end face of the compressor is not necessarily the only structure which transmits the force; other portions of the compressor, in particular the laterally facing surfaces of the compressor, also can transmit some of the force F.

FIGS. 8B-8D illustrate yet further aspects of the present invention. More specifically, the directions of views of the Doppler crystals can be substantially parallel (FIG. 8B), divergent or convergent (FIG. 8C), or combinations of parallel and di-/convergent directions of view (FIG. 8D).

Methods and devices embodying features of the invention may be used to occlude any artery; in the following discussion, the uterine artery is used as an example. It will be understood that the methods and devices discussed in regard to this example may also be applied to any other artery, particularly any other artery located near a body wall such as a vaginal wall, a rectal wall, and abdominal wall, skin surface, or other body surface.

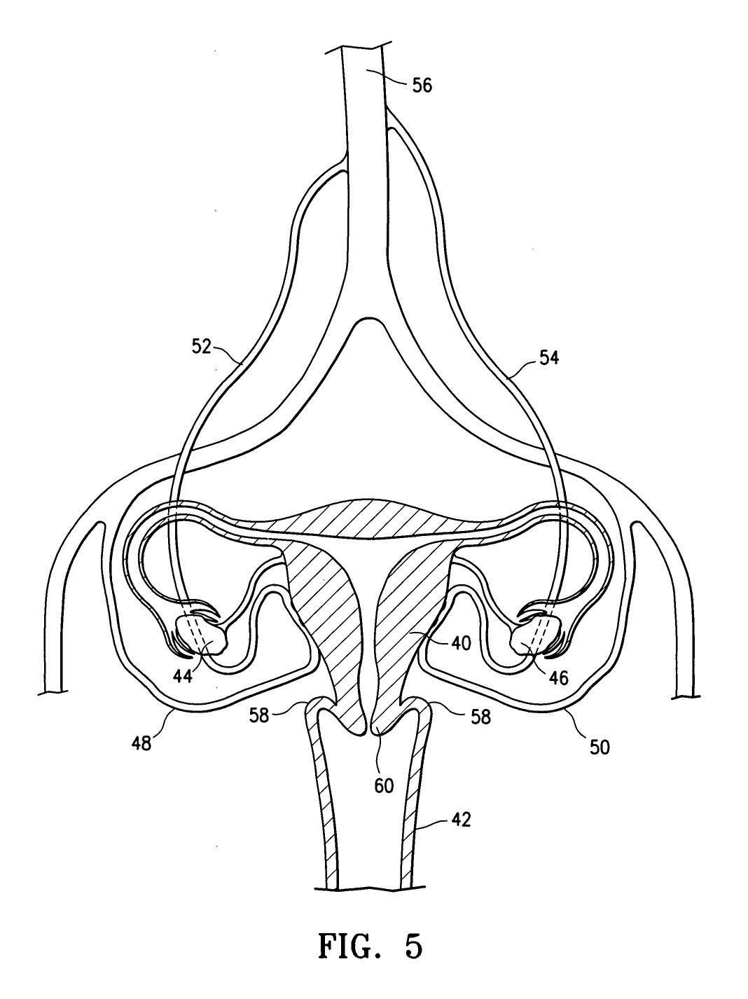

FIG. 5 illustrates a typical human female reproductive system, including a uterus 40, vagina 42, right ovary 44, and left ovary 46. Blood is supplied to the uterus 40 primarily via the right uterine artery 48 and the left uterine artery 50, and secondarily via the right ovarian artery 52 and the left ovarian artery 54, all of which are supplied by the aorta 56. Note the close apposition of the uterine arteries 48 and 50 to the vaginal fornix 58 and to the uterine cervix 60.

A method of occluding an artery includes sensing an artery, and compressing an artery with a clamping device having a blood flow sensor. Sensing an artery may include sensing blood flow, such as blood flow in an artery. Compressing an artery may include grasping tissue near to an artery, and may include compressing tissue surrounding an artery effective to compress the artery.

One method of occluding a uterine artery includes applying an artery occluding device to the artery so that blood flow through the artery is reduced, or is abolished. Such occlusion is effected by clamping an artery such as a uterine artery. Clamping of a uterine artery preferably is accomplished by applying a clamping device to tissue near to a uterine artery effective to compress the uterine artery.

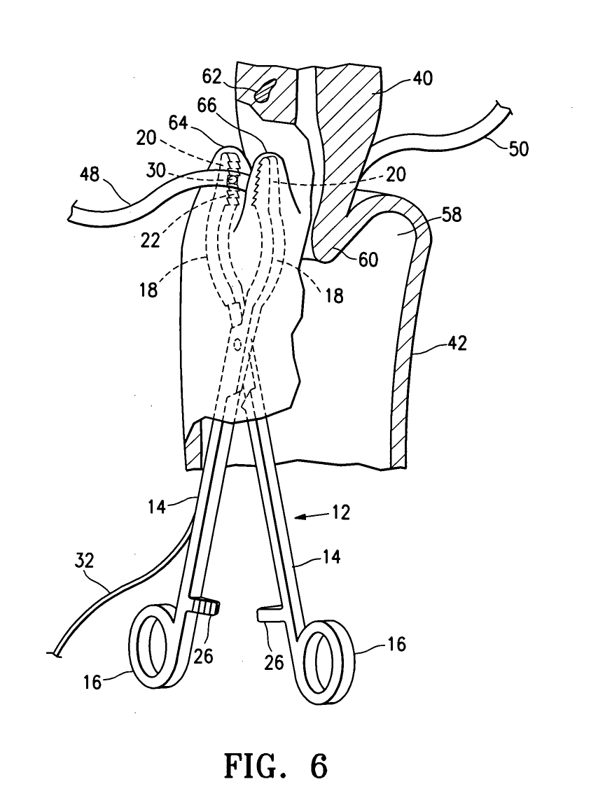

FIG. 6 illustrates the use of a non-invasive artery occluding device embodying features of the invention. A vaginal clamp 12 (the clamping component of a non-invasive artery occluding system 10, only parts of which are illustrated in FIG. 6) is shown partially within a vagina 42 of a female patient having a uterus 40 with a uterine fibroid 62 (one of the several medical conditions which may be treated by occlusion of the uterine arteries). The uterine arteries 48 and 50 approach the uterus 40 not far from the vaginal fornix 58 and the uterine cervix 60. The vaginal clamp 12 has handles 14 with finger holes 16, and pressure-applying members 18 with jaws 20 having tissue-contacting surfaces 22. The vaginal clamp 12 also includes a sensor 30 on a jaw 20 facing the patient's tissue, and communicating with other parts of the system 10 (not shown in the Figure) via a cable 32.

A uterine artery may be accessed via the vagina of a patient, and compressing a uterine artery may be accomplished by compressing a portion of the vaginal wall around a portion of a uterine artery. The vaginal clamp is able to access the uterine arteries via the vagina 42, by pressing with jaws 20 on the vaginal wall near the vaginal fornix 58 so as to distend portions 64 and 66 of the vaginal wall to more closely approach the right uterine artery 48. Pressure from jaws 20 is thus effective to invaginate the vaginal wall in order to bring tissue around uterine artery 48 as shown in FIG. 6. Sensor 30 is effective to detect the presence of and to locate uterine artery 48, and to detect blood flow in the artery 48. Sensor 30 is used to aid in positioning jaws 20 and tissue-contacting surfaces 22 to best surround uterine artery 48 by vaginal wall portions 64 and 66 and associated tissue. Closing jaws 20 presses tissue-contacting surfaces 22 more strongly into the vaginal wall portions 64 and 66, compressing uterine artery 48 and other tissue between the jaws 20, effective to occlude uterine artery 48. Sensor 30 is be used to detect the resulting reduction or abolition of blood flow in uterine artery 48, and to adjust the amount of pressure or force used in order to effect the desired amount of reduction in blood flow and to confirm abolition of blood flow if desired. A locking mechanism 26 may be used to maintain the desired amount of pressure or force on the tissue for a desired amount of time. Blood flow in the left uterine artery 50 may be similarly occluded, by the same vaginal clamp 12 (after release of the occlusion of the right uterine artery 48) or by a different vaginal clamp 12 (thus allowing simultaneous clamping and occlusion of both uterine arteries).

A method of occluding uterine arteries includes occluding both uterine arteries simultaneously by inserting the pressure applying members along the exterior of the cervix and to the vaginal fornix and compressing both arteries against the body of the uterus.

A clamping device suitable for use in a method embodying features of the invention has a releasable clamping device, so that a uterine artery remains occluded for only a limited time. A suitable limited time may be between about 0.2 hours and about 12 hours, or preferably between about 0.5 hours and about 4 hours.

Non-invasive artery occluding devices embodying features of the invention may be made from any suitable material or combination of materials, including metals such as stainless steel and shape memory alloys such as nickel titanium alloys, plastics, ceramics, and other materials known in the art. Biocompatible polymers, such as for example, polycarbonate, polysulfone, polyester, polyacetal, and other polymers may be particularly suitable for embodiments of the invention. The device or system may be designed for single use (disposable) or may be sterilizable and capable of multiple use.

While particular forms of the invention have been illustrated and described herein, it will be apparent that various modifications and improvements can be made to the invention. Moreover, individual features of embodiments of the invention may be shown in some drawings and not in others, but those skilled in the art will recognize that individual features of one embodiment of the invention can be combined with any or all the features of another embodiment. Accordingly, it is not intended that the invention be limited to the specific embodiments illustrated. It is therefore intended that this invention be defined by the scope of the appended claims as broadly as the prior art will permit.

Terms such as “element”, “member”, “device”, “sections”, “portion”, “section”, “steps” and words of similar import when used herein shall not be construed as invoking the provisions of 35 U.S.C §112(6) unless the following claims expressly use the terms “means” or “step” followed by a particular function without reference to a specific structure or action. All patents and all patent applications referred to above are hereby incorporated by reference in their entirety.

What is claimed is:

1. A non-invasive uterine artery occlusion device for treating adenomyosis and endometriosis, comprising:
a. a pair of pressure-applying members having opposed tissue-contacting surfaces on distal portions thereof;
b. a pair of pivotally-connected supporting shafts extending from said pair of pressure-applying members configured to adjust the distance between the opposed tissue-contacting surfaces of the pressure-applying members; and
c. at least one sensor for locating a uterine artery disposed in or on a pressure-applying member to facilitate location of the uterine artery to be occluded.

2. The non-invasive uterine artery occlusion device of claim 1, wherein said at least one sensor is selected from the group of sensors consisting of blood flow sensors, sound sensors, pressure sensors, strain sensors, stress sensors, chemical sensors, electromagnetic radiation sensors, and combinations thereof.

3. The non-invasive uterine artery occlusion device of claim 2, wherein said sensor comprises a blood flow sensor.

4. The non-invasive uterine artery occlusion device of claim 3, wherein said blood flow sensor comprises a Doppler ultrasound sensor.

5. The non-invasive uterine artery occlusion device of claim 4, wherein said Doppler sensor is configured to sense ultrasound energy having a frequency of between about 5 MHz and about 19 MHz.

6. The non-invasive uterine artery occlusion device of claim 5, wherein said Doppler ultrasound sensor is configured to sense ultrasound energy having a frequency of between about 6 MHz and about 10 MHz.

7. The non-invasive uterine artery occlusion device of claim 6, wherein said Doppler ultrasound sensor is configured to sense ultrasound energy having a frequency of about 8 MHz.

8. The non-invasive uterine artery occlusion device of claim 2, wherein said sensor is an electromagnetic radiation sensor configured to sense electromagnetic radiation having a wavelength of between about 500 nanometers (nm) and about 2000 nm.

9. The non-invasive uterine artery occlusion device of claim 2, wherein said sensor is an electromagnetic radiation sensor configured to sense electromagnetic radiation having a wavelength of between about 700 nm and about 1000 nm.

10. The device of claim 1, wherein at least one sensor has a sensing direction effective that a uterine artery disposed along said sensing direction will likely be detected, and wherein said at least one sensor is disposed on said tissue-contacting surface effective that said sensing direction is perpendicular to said tissue-contacting surface.

11. The device of claim 1, wherein a pressure-applying member has a distal tip and wherein said sensor is spaced between about 0.1 inch and about 1 inch proximal of said distal tip.

12. The device of claim 11, wherein said sensor is disposed at a location between about 0.2 inch and about 0.6 inch proximal of said distal tip.

13. The device of claim 12, wherein said sensor is disposed at a location about 0.4 inch proximal of said distal tip.

14. The device of claim 1, wherein said non-invasive uterine artery occlusion device is configured for intravaginal use.

15. The non-invasive uterine artery occlusion device of claim 11, further comprising a pair of handles with a tip, each of said handles connected to one of said supporting shafts, and wherein said each of said handle tips is separated from said distal tip of said pressure-applying member by a length of between about 0.5 inch and about 16 inches.

16. The non-invasive uterine artery occlusion device of claim 15, wherein said length comprises a length of between about 1 inch and about 12 inches.

17. The non-invasive uterine artery occlusion device of claim 1, further comprising a locking mechanism configured to retain a pressure-applying member in a desired position.

18. The non-invasive uterine artery occlusion device of claim 17, wherein said locking mechanism comprises a releasable mechanism.

19. The non-invasive uterine artery occlusion device of claim 1, wherein said pair of opposed pressure-applying members are movably configured effective to compress tissue disposed between said pressure-applying members upon movement of said pressure-applying members.

20. The non-invasive uterine artery occlusion device of claim 1, wherein said pressure-applying members are configured to apply between about 15 pounds per square inch (psi) of pressure and about 125 psi of pressure to tissue disposed between said pressure-applying members.

21. The non-invasive uterine artery occlusion device of claim 20, wherein said pressure-applying members are configured to apply between about 30 psi of pressure and about 60 psi of pressure to tissue disposed between said pressure-applying members.

22. The non-invasive uterine artery occlusion device of claim 1, comprising a plurality of sensors.

23. The non-invasive uterine artery occlusion device of claim 22 having at least one sensor on both pressure applying members.

24. A system comprising:
a non-invasive uterine artery occlusion device for treating adenomyosis and endometriosis comprising a pair of pressure-applying members having opposed tissue-contacting surfaces on distal portions thereof; a pair of pivotally-connected supporting shafts extending from said pair of pressure-applying members configured to adjust the distance between the opposed tissue-contacting surfaces of the pressure-applying members; and at least one sensor for locating a uterine artery disposed in or on a pressure-applying member; and
a sensor controller operatively connected to said sensor and comprising a source of power.

25. The system of claim 24, wherein said sensor comprises a Doppler ultrasound sensor and said sensor controller comprises a Doppler ultrasound controller.

26. The system of claim 24, wherein said sensor controller is configured to provide an output detectable by an operator.

27. The system of claim 24, further comprising a device configured for grasping a portion of a patient's body.

28. The system of claim 27, wherein said device for grasping a portion of a patient's body comprises a device configured for grasping a uterine cervix.

29. A method of treating adenomyosis and endometriosis by occluding a uterine artery of a female patient, comprising:
a.) providing a uterine artery occluding device having a pressure applying surface and a blood flow sensor.
b.) locating a uterine artery with the blood flow sensor; and
c.) occluding a portion of said uterine artery by pressuring the pressure applying surface of the occluding device to tissue adjacent the uterine artery with the occluding device.

30. The method of claim 29, wherein said sensor comprises a blood flow sensor.

31. The method of claim 30, wherein said blood flow sensor comprises a Doppler ultrasound blood flow sensor.

32. The method of claim 30, further comprising detecting a change in blood flow in said uterine artery.

33. The method of claim 30, wherein said locating comprises detecting blood flow in a uterine artery.

34. The method of claim 33, wherein compressing comprises applying pressure to a vaginal wall.

35. The method of claim 34, further comprising grasping a uterine cervix.

36. The method of claim 33, wherein said non-invasive uterine artery occlusion device comprises a releasable non-invasive uterine artery occlusion device, and said uterine artery remains occluded for only a limited time.

37. The method of claim 36, wherein said limited time comprises a time of between about 0.2 hours and about 12 hours.

38. The method of claim 36, wherein said limited time comprises a time of between about 0.5 hours and about 4 hours.

39. The method of claim 33, wherein said compressing a portion of said uterine artery comprises applying between about 15 psi of pressure and about 125 psi of pressure to body tissue.

40. The method of claim 33, wherein said compressing a portion of said uterine artery comprises applying between about 30 psi of pressure and about 60 psi of pressure to body tissue.

41. The method of claim 30, wherein said locating comprises detecting blood flow in a uterine artery with a blood flow sensor disposed on a pressure-applying member with a tissue-contacting surface defining a direction opposite said surface, said blood flow sensor being configured to locate a uterine artery occupying a location disposed in a direction substantially opposite said tissue-contacting surface.

42. The method of claim 31, wherein locating with said Doppler ultrasound blood flow sensor comprises locating a uterine artery with ultrasound having a frequency of between about 5 MHz and about 19 MHz.

43. The method of claim 31, wherein locating with said Doppler ultrasound blood flow sensor comprises locating a uterine artery with ultrasound having a frequency of between about 6 MHz and about 10 MHz.

44. The method of claim 31, wherein locating with said Doppler ultrasound blood flow sensor comprises locating a uterine artery with ultrasound having a frequency of about 8 MHz.

45. The method of claim 29 wherein both uterine arteries are simultaneously occluded.

46. A non-invasive uterine artery occlusion device for treating adenomyosis and endometriosis, comprising:
a. a pair of pressure-applying members having opposed tissue-contacting surfaces on distal portions thereof;
b. a pair of pivotally-connected supporting shafts extending from said pair of pressure-applying members configured to adjust the distance between the opposed tissue-contacting surfaces of the pressure-applying members; and
c. an ultrasound sensor/transducer for locating a uterine artery disposed in or on a pressure-applying member configured to provide ultrasound signals and to receive ultrasound reflections so as to facilitate location of the uterine artery to be occluded.

47. A non-invasive uterine artery occlusion device for treating adenomyosis and endometriosis, comprising:
a. pressure-applying means for applying pressure to tissue;
b. sensor means for locating a uterine artery to facilitate location of the uterine artery to be occluded;
and
c. supporting means for supporting said pressure-applying means and said sensor means.

48. A non-invasive uterine artery occlusion device for treating adenomyosis and endometriosis, comprising:
a. pressure-applying means for applying pressure to tissue;
b. sensor/transducer means for providing signals and for sensing reflected signals for locating a uterine artery to facilitate location of the uterine artery to be occluded;
and
c. supporting means for supporting said pressure-applying means and said sensor/transducer means.
